(19) United States
(12) Patent Application Publication
(10) Pub. No.: US 2012/0029595 A1
(43) Pub. Date: Feb. 2, 2012

(54) Bilateral Sound Processor Systems and Methods

(75) Inventors: Tracey Kruger, Valencia, CA (US); Guillermo A. Calle, Moorpark, CA (US)

(73) Assignee: Advanced Bionics AG, c/o Froriep Renggli, Zug (CH)

(21) Appl. No.: 12/847,115

(22) Filed: Jul. 30, 2010

(51) Int. Cl. A61F 11/04; A61N 1/36

(52) U.S. Cl. 607/57

(57) ABSTRACT

An exemplary sound processor includes a storage facility configured to maintain data representative of a first program set associated with a first cochlear implant and data representative of a second program set associated with a second cochlear implant, a detection facility configured to detect when the sound processor is communicatively coupled to the first cochlear implant and to detect when the sound processor is communicatively coupled to the second cochlear implant, and an operation facility configured to operate in accordance with the first program set in response to a detection that the sound processor is communicatively coupled to the first cochlear implant and to operate in accordance with the second program set in response to a detection that the sound processor is communicatively coupled to the second cochlear implant. Corresponding methods and systems are also described.

### BACKGROUND INFORMATION

The natural sense of hearing in human beings involves the use of hair cells in the cochlea that convert or transduce acoustic signals into auditory nerve impulses. Hearing loss, which may be due to many different causes, is generally of two types: conductive and sensorineural. Conductive hearing loss occurs when the normal mechanical pathways for sound to reach the hair cells in the cochlea are impeded. These sound pathways may be impeded, for example, by damage to the auditory ossicles. Conductive hearing loss may often be overcome through the use of conventional hearing aids that amplify sound so that acoustic signals can reach the hair cells within the cochlea. Some types of conductive hearing loss may also be treated by surgical procedures.

Sensorineural hearing loss, on the other hand, is caused by the absence or destruction of the hair cells in the cochlea, which are needed to transduce acoustic signals into auditory nerve impulses. People who suffer from sensorineural hearing loss may be unable to derive significant benefit from conventional hearing aid systems, no matter how loud the acoustic stimulus. This is because the mechanism for transducing sound energy into auditory nerve impulses has been damaged. Thus, in the absence of properly functioning hair cells, auditory nerve impulses cannot be generated directly from sounds.

To overcome sensorineural hearing loss, numerous cochlear implant systems—or cochlear prostheses—have been developed. Cochlear implant systems bypass the hair cells in the cochlea by presenting electrical stimulation directly to the auditory nerve fibers by way of one or more channels formed by an array of electrodes implanted in the cochlea. Direct stimulation of the auditory nerve fibers leads to the perception of sound in the brain and at least partial restoration of hearing function.

Cochlear implant patients rely on the uptime and availability of their cochlear implant system hardware in order to maintain their sense of hearing. However, the reliability of a patient's external cochlear implant system equipment, such as a sound processor, may be limited. In addition, a patient's sound processor may be subject to damage, theft, or loss. As a result, a cochlear implant patient may keep a secondary sound processor that can be used in place of a primary sound processor in the event that the primary sound processor is unavailable. However, sound processors are very expensive, so this redundancy comes at a cost to the patient. This problem is even worse for bilateral patients (i.e., patients with two cochlear implants) who heretofore have had to keep two secondary sound processors on hand.

### SUMMARY

An exemplary sound processor includes a storage facility configured to maintain data representative of a first program set associated with a first cochlear implant and data representative of a second program set associated with a second cochlear implant, a detection facility configured to detect when the sound processor is communicatively coupled to the first cochlear implant and to detect when the sound processor is communicatively coupled to the second cochlear implant, and an operation facility configured to operate in accordance with the first program set in response to a detection that the sound processor is communicatively coupled to the first cochlear implant and to operate in accordance with the second program set in response to a detection that the sound processor is communicatively coupled to the second cochlear implant.

Another exemplary sound processor includes a storage facility configured to maintain data representative of a first program set associated with a first cochlear implant and data representative of a second program set associated with a second cochlear implant, a communication facility configured to selectively communicate with the first cochlear implant and the second cochlear implant, a detection facility configured to detect when the sound processor is communicatively coupled to the first cochlear implant and to detect when the sound processor is communicatively coupled to the second cochlear implant, and an operation facility configured to process audio signals in accordance with the first program set in response to a detection by the detection facility that the sound processor is communicatively coupled to the first cochlear implant and to process audio signals in accordance with the second program set in response to a detection by the detection facility that the sound processor is communicatively coupled to the second cochlear implant.

An exemplary method includes a sound processor maintaining data representative of a first program set associated with a first cochlear implant and data representative of a second program set associated with a second cochlear implant, detecting a communicative coupling of the sound processor to the first cochlear implant, and operating in accordance with the first program set in response to the detecting of the communicative coupling to the first cochlear implant.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 illustrates an exemplary cochlear implant system according to principles described herein.

FIG. 2 illustrates an exemplary cochlear implant fitting system according to principles described herein.

FIG. 3 illustrates exemplary components of an exemplary fitting subsystem according to principles described herein.

FIG. 4 illustrates exemplary components of an exemplary sound processor according to principles described herein.

FIG. 5 illustrates an exemplary implementation of the cochlear implant fitting system of FIG. 2 according to principles described herein.

FIG. 6 illustrates an exemplary method of operation of the exemplary sound processor of FIG. 4 according to principles described herein.

FIG. 7 illustrate an exemplary loading of data representative of multiple program sets onto a sound processor according to principles described herein.

FIG. 8 illustrates an exemplary communicative coupling of a sound processor to a first cochlear implant of a bilateral cochlear implant patient according to principles described herein.

FIG. 9 illustrates an exemplary communicative coupling of the sound processor of FIG. 8 to a second cochlear implant of the bilateral cochlear implant patient of FIG. 8 according to principles described herein.

FIG. 10 illustrates an exemplary computing device according to principles described herein.

### DETAILED DESCRIPTION

Bilateral sound processor systems and methods are described herein. As described in more detail below, a sound processor may be configured to maintain a first program set associated with a first cochlear implant and a second program set associated with a second cochlear implant. The sound processor may be configured to detect a communicative coupling to either of the first or second cochlear implants and operate in accordance with the program set associated with the cochlear implant to which the sound processor is communicatively coupled. Accordingly, for example, the sound processor can dynamically adapt to multiple cochlear implants and properly process audio signals regardless of the cochlear implant to which the sound processor is communicatively coupled.

Numerous advantages may be associated with the methods and systems described herein. For example, a bilateral cochlear implant patient may selectively use a single sound processor with either her left or right cochlear implant. This may allow the bilateral cochlear implant patient to switch a single sound processor from one cochlear implant to another (e.g., from a nondominant ear to a dominant ear) to compensate for a sound processor that is lost, damaged, stolen, or otherwise unavailable (e.g., has a dead battery). Additionally or alternatively, a bilateral cochlear implant patient may keep a single secondary sound processor as a backup for both of the patient's primary sound processors, thereby reducing the cost to the patient.

As used herein, the term “program set” refers to any program or combination of programs (e.g., sound processing programs) executable by a sound processor included in a cochlear implant system. Hence, a program set may specify a particular mode in which the sound processor is to operate and/or define a set of control parameters selected to optimize a listening experience of a cochlear implant patient. In some examples, a program set may be configured to facilitate measurement of one or more electrode impedances, performance of one or more neural response detection operations, and/or performance of one or more diagnostics procedures associated with the cochlear implant system. A fitting subsystem may adjust one or more control parameters associated with a particular program set in response to patient feedback and/or user input in order to customize the particular program set to a particular cochlear implant of the patient.

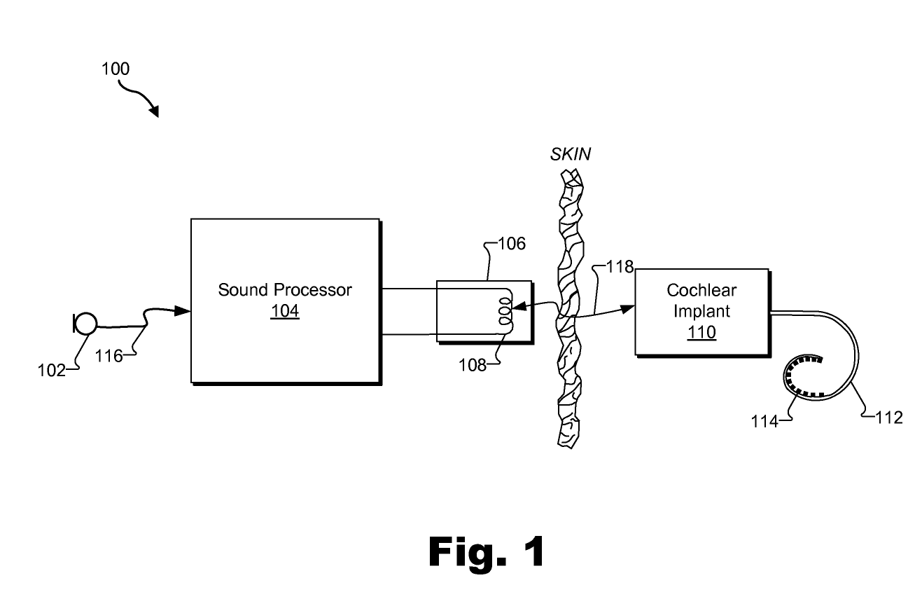

To facilitate an understanding of the methods and systems described herein, an exemplary cochlear implant system 100 will be described in connection with FIG. 1. As shown in FIG. 1, cochlear implant system 100 may include a microphone 102, a sound processor 104, a headpiece 106 having a coil 108 disposed therein, a cochlear implant 110 (also referred to as an “implantable cochlear stimulator”), and a lead 112 with a plurality of electrodes 114 disposed thereon. Additional or alternative components may be included within cochlear implant system 100 as may serve a particular implementation.

As shown in FIG. 1, microphone 102, sound processor 104, and headpiece 106 may be located external to a cochlear implant patient. In some alternative examples, microphone 102 and/or sound processor 104 may be implanted within the patient. In such configurations, the need for headpiece 106 may be obviated.

Microphone 102 may detect an audio signal and convert the detected signal to a corresponding electrical signal. The electrical signal may be sent from microphone 102 to sound processor 104 via a communication link 116, which may include a telemetry link, a wire, and/or any other suitable communication link.

Sound processor 104 is configured to direct cochlear implant 110 to generate and apply electrical stimulation (also referred to herein as “stimulation current”) to one or more stimulation sites within a cochlea of the patient. To this end, sound processor 104 may process the audio signal detected by microphone 102 in accordance with a selected sound processing strategy to generate appropriate stimulation parameters for controlling cochlear implant 110. Sound processor 104 may include or be implemented by a behind-the-ear (“BTE”) unit, a portable speech processor (“PSP”), and/or any other sound-processing unit as may serve a particular implementation. Exemplary components of sound processor 104 will be described in more detail below.

Sound processor 104 may be configured to transcutaneously transmit, in accordance with a program set associated with cochlear implant 110, one or more control parameters and/or one or more power signals to cochlear implant 110 with coil 108 by way of a communication link 118. These control parameters may be configured to specify one or more stimulation parameters, operating parameters, and/or any other parameter by which cochlear implant 110 is to operate as may serve a particular implementation. Exemplary control parameters include, but are not limited to, stimulation current levels, volume control parameters, program selection parameters, operational state parameters (e.g., parameters that turn a sound processor and/or a cochlear implant on or off), audio input source selection parameters, fitting parameters, noise reduction parameters, microphone sensitivity parameters, microphone direction parameters, pitch parameters, timbre parameters, sound quality parameters, most comfortable current levels (“M levels”), threshold current levels (“T levels”), channel acoustic gain parameters, front and backend dynamic range parameters, current steering parameters, pulse rate values, pulse width values, frequency parameters, amplitude parameters, waveform parameters, electrode polarity parameters (i.e., anode-cathode assignment), location parameters (i.e., which electrode pair or electrode group receives the stimulation current), stimulation type parameters (i.e., monopolar, bipolar, or tripolar stimulation), burst pattern parameters (e.g., burst on time and burst off time), duty cycle parameters, spectral tilt parameters, filter parameters, and dynamic compression parameters. Sound processor 104 may also be configured to operate in accordance with one or more of the control parameters.

As shown in FIG. 1, coil 108 may be housed within headpiece 106, which may be affixed to a patient's head and positioned such that coil 108 is communicatively coupled to a corresponding coil included within cochlear implant 110. In this manner, control parameters and power signals may be wirelessly transmitted between sound processor 104 and cochlear implant 110 via communication link 118. It will be understood that data communication link 118 may include a bi-directional communication link and/or one or more dedicated uni-directional communication links. In some alternative embodiments, sound processor 104 and cochlear implant 110 may be directly connected with one or more wires or the like.

Cochlear implant 110 may be configured to generate electrical stimulation representative of an audio signal detected by microphone 102 in accordance with one or more stimulation parameters transmitted thereto by sound processor 104. Cochlear implant 110 may be further configured to apply the electrical stimulation to one or more stimulation sites within the cochlea via one or more electrodes 114 disposed along lead 112. In some examples, cochlear implant 110 may include a plurality of independent current sources each associated with a channel defined by one or more of electrodes 114. In this manner, different stimulation current levels may be applied to multiple stimulation sites simultaneously by way of multiple electrodes 114. In such examples, cochlear implant system 100 may be referred to as a “multi-channel cochlear implant system.”

To facilitate application of the electrical stimulation generated by cochlear implant 110, lead 112 may be inserted within a duct of the cochlea such that electrodes 114 are in communication with one or more stimulation sites within the cochlea. As used herein, the term “in communication with” refers to electrodes 114 being adjacent to, in the general vicinity of, in close proximity to, directly next to, or directly on the stimulation site. Any number of electrodes 114 (e.g., sixteen) may be disposed on lead 112 as may serve a particular implementation.

In certain examples, cochlear implant 110, a corresponding program set, and/or a corresponding cochlear implant patient may be associated with a unique identifier (e.g., a unique serial number) stored within cochlear implant 110. The unique identifier may be configured to distinguish cochlear implant 110, a corresponding program set, and/or a corresponding cochlear implant patient from other cochlear implants, program sets, and/or cochlear implant patients. In some examples, the unique identifier may be detectable by sound processor 104 and/or other devices (e.g., by a fitting station) communicatively coupled to cochlear implant 110 and used to identify cochlear implant 110. As will be explained in more detail below, sound processor 104 may be configured to detect the unique identifier to identify cochlear implant 110 and selectively operate in accordance with a specific program set associated with cochlear implant 110 based on the identification of cochlear implant 110.

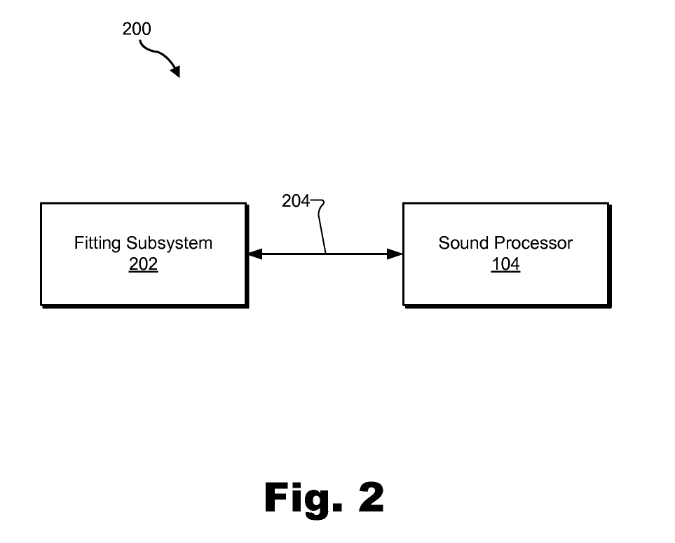

FIG. 2 illustrates an exemplary cochlear implant fitting system 200 (or simply “fitting system 200”) that may be used to fit sound processor 104 to a patient. As used herein, the terms “fitting a sound processor to a patient” and “fitting a cochlear implant system to a patient” will be used interchangeably to refer to performing one or more fitting operations associated with sound processor 104 and/or any other component of cochlear implant system 100. Such fitting operations may include, but are not limited to, adjusting one or more control parameters by which sound processor 104 and/or cochlear implant 110 operate, measuring one or more electrode impedances, performing one or more neural response detection operations, and/or performing one or more diagnostics procedures associated with the cochlear implant system.

As shown in FIG. 2, fitting system 200 may include a fitting subsystem 202 configured to be selectively and communicatively coupled to sound processor 104 of cochlear implant system 100 by way of a communication link 204. Fitting subsystem 202 and sound processor 104 may communicate using any suitable communication technologies, devices, networks, media, and protocols supportive of data communications.

Fitting subsystem 202 may be configured to perform one or more of the fitting operations described herein. To this end, fitting subsystem 202 may be implemented by any suitable combination of computing and communication devices including, but not limited to, a fitting station, a personal computer, a laptop computer, a handheld device, a mobile device (e.g., a mobile phone), a clinician's programming interface (“CPI”) device, and/or any other suitable component as may serve a particular implementation. An exemplary implementation of fitting subsystem 202 will be described in more detail below.

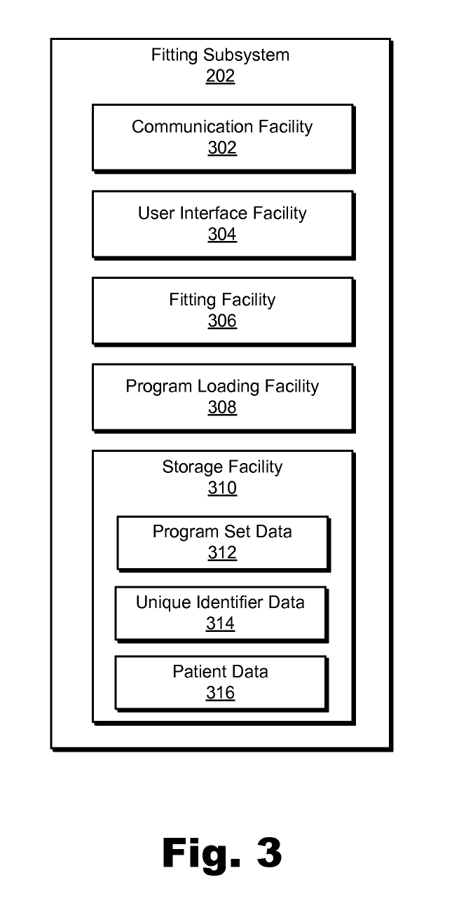

FIG. 3 illustrates exemplary components of fitting subsystem 202. As shown in FIG. 3, fitting subsystem 202 may include a communication facility 302, a user interface facility 304, a fitting facility 306, a program loading facility 308, and a storage facility 310, which may be communicatively coupled to one another using any suitable communication technologies. Each of these facilities will now be described in more detail.

Communication facility 302 may be configured to facilitate communication between fitting subsystem 202 and sound processor 104. For example, communication facility 302 may be implemented by a CPI device, which may include any suitable combination of components configured to allow fitting subsystem 202 to interface and communicate with sound processor 104. Communication facility 302 may additionally or alternatively include one or more transceiver components configured to wirelessly transmit data (e.g., program data and/or control parameter data) to sound processor 104 and/or wirelessly receive data (e.g., feedback data, impedance measurement data, neural response data, etc.) from sound processor 104.

Communication facility 302 may additionally or alternatively be configured to facilitate communication between fitting subsystem 302 and one or more other devices. For example, communication facility 302 may be configured to facilitate communication between fitting subsystem 302 and one or more computing devices (e.g., by way of the Internet and/or one or more other types of networks), reference implants, and/or any other computing device as may serve a particular implementation.

User interface facility 304 may be configured to provide one or more user interfaces configured to facilitate user interaction with fitting subsystem 202. For example, user interface facility 304 may provide a graphical user interface (“GUI”) through which one or more functions, options, features, and/or tools associated with one or more fitting operations described herein may be provided to a user and through which user input may be received. In certain embodiments, user interface facility 304 may be configured to provide the GUI to a display device (e.g., a computer monitor) for display.

Fitting facility 306 may be configured to perform one or more of the fitting operations described herein. For example, fitting facility 306 may be configured to adjust one or more control parameters by which sound processor 104 and/or cochlear implant 110 operate, direct sound processor 104 to measure one or more electrode impedances, perform one or more neural response detection operations, and/or perform one or more diagnostics procedures associated with cochlear implant system 100.

In some examples, fitting facility 306 may be configured to selectively use one or more programs sets that have been loaded onto sound processor 104 to fit sound processor 104 to a patient. The loading of the one or more program sets may be performed by program loading facility 308, as will be described inmore detail below. In some examples, fitting facility 306 may be configured to use a first program set to fit sound processor 104 to a first cochlear implant associated with a first ear of a patient and a second program set to fit sound processor 104 to a second cochlear implant associated with a second ear of the patient.

In some examples, fitting facility 306 may be configured to initialize sound processor 104 prior to fitting sound processor 104 to a patient. Such initialization may include, but is not limited to, associating sound processor 104 with a particular patient (e.g., associating sound processor 104 with patient-specific fitting data and/or associating sound processor 104 with one or more unique identifiers associated with the patient), associating sound processor 104 with one or more particular cochlear implants 110 (e.g., associating sound processor 104 with one or more unique identifiers associated with the one or more particular cochlear implants 110), loading data onto sound processor 104, clearing data from sound processor 104, and/or otherwise preparing sound processor 104 for a fitting session in which sound processor 104 is to be fitted to a patient.

Program loading facility 308 may be configured to load data representative of one or more programs sets onto sound processor 104 for use by sound processor 104 during and/or after a fitting session. In some examples, program loading facility 308 may be configured to load program data representative of a plurality of program sets onto sound processor 104 during a data transfer or fitting session. In this manner, a user (e.g., an audiologist) of fitting subsystem 202 may direct sound processor 104 to switch between multiple program sets during a fitting session (e.g., to fit sound processor 104 to multiple cochlear implants).

In some examples, program loading facility 308 may be configured to load program data representative of a plurality of program sets onto sound processor 104 by transmitting the program data to sound processor 104 and directing sound processor to cache the program data as a library of program sets in a storage medium (e.g., memory) included within sound processor 104. The program data may include any type of data (e.g., digital signal processing (“DSP”) code) and may be cached within sound processor 104 for any amount of time as may serve a particular implementation.

Program loading facility 308 may be implemented by a fitting station and/or other computing device utilized by a clinician or other user to fit sound processor 104 to a patient. In this manner, the loading of the program data may be performed during an initialization of sound processor 104 and/or at any point during or after a fitting session in which sound processor 104 is fit to the patient.

Storage facility 310 may be configured to maintain program set data 312 representative of one or more program sets, unique identifier data 314 representative of one or more unique identifiers, and patient data 316 representative of data descriptive of or otherwise associated with one or more cochlear implant patients. Storage facility 310 may be configured to maintain additional or alternative data as may serve a particular implementation.

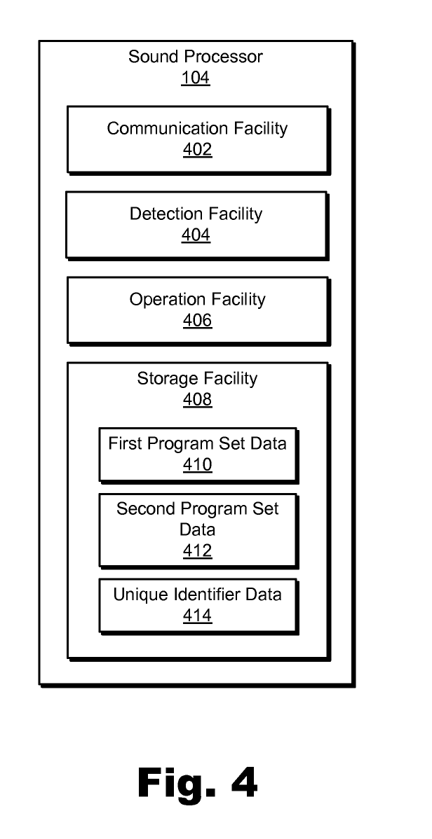

FIG. 4 illustrates exemplary components of sound processor 104. As shown in FIG. 4, sound processor 104 may include a communication facility 402, a detection facility 404, an operation facility 406, and a storage facility 408, any or all of which may be in communication with one another using any suitable communication technologies. Each of these facilities will now be described in more detail.

Communication facility 402 may be configured to facilitate communication between sound processor 104 and fitting subsystem 202 and/or cochlear implant 110. For example, communication facility 402 may be configured to facilitate a communicative coupling of sound processor 104 to a CPI device in order to communicate with fitting subsystem 202. Communication facility 402 may be further configured to facilitate a communicative coupling of sound processor 104 to cochlear implant 110. For example, communication facility 402 may include transceiver components configured to wirelessly transmit data (e.g., program set data including control parameters and/or power signals) to cochlear implant 110 and/or wirelessly receive data (e.g., unique identifier data) from cochlear implant 110.

Detection facility 404 may be configured to detect when sound processor 104 is communicatively coupled to one or more cochlear implants. For example, detection facility 404 may be configured to detect when sound processor 104 is communicatively coupled to a first cochlear implant and detect when sound processor 104 is communicatively coupled to a second cochlear implant. The detection may be made in any suitable way. In some examples, detection facility 404 may be configured to detect the transmission/receipt of signals to/from a cochlear implant. Additionally or alternatively, detection facility 404 may be configured to detect a first unique identifier associated with the first cochlear implant and identify the first cochlear implant based on the first unique identifier and detect a second unique identifier associated with the second cochlear implant and identify the second cochlear implant based on the second unique identifier, as will be explained in more detail below.

Operation facility 406 may be configured to perform one or more signal processing heuristics on an audio signal presented to the patient. For example, operation facility 406 may perform one or more pre-processing operations, spectral analysis operations, noise reduction operations, mapping operations, and/or any other types of signal processing operations on a detected audio signal as may serve a particular implementation. In some examples, operation facility 406 may generate and/or adjust one or more control parameters governing an operation of cochlear implant 110 (e.g., one or more stimulation parameters defining the electrical stimulation to be generated and applied by cochlear implant 110). In some examples, operation facility 406 may be configured to operate in accordance with one or more program sets provided by fitting subsystem 202 and/or otherwise stored within storage facility 408.

For example, operation facility 406 may be configured to operate in accordance with a first program set associated with a first cochlear implant in response to a detection (e.g., a detection by detection facility 404) that sound processor 104 is communicatively coupled to the first cochlear implant. Similarly, operation facility 406 may be configured to operate in accordance with a second program set associated with a second cochlear implant in response to a detection that sound processor 104 is communicatively coupled to the second cochlear implant. Accordingly, operation facility 406 may be configured to dynamically adapt its operation depending on the cochlear implant to which sound processor 104 is communicatively coupled, as will be explained in more detail below.

Storage facility 408 may be configured to maintain first program set data 410 representative of a first program set associated with a first cochlear implant, second program set data 412 representative of a second program set associated with a second cochlear implant, and unique identifier data 414 representative of one or more unique identifiers (e.g., a first unique identifier associated with the first cochlear implant and/or the first program set and a second unique identifier associated with the second cochlear implant and/or the second program set). Storage facility 408 may be configured to maintain additional or alternative data as may serve a particular implementation.

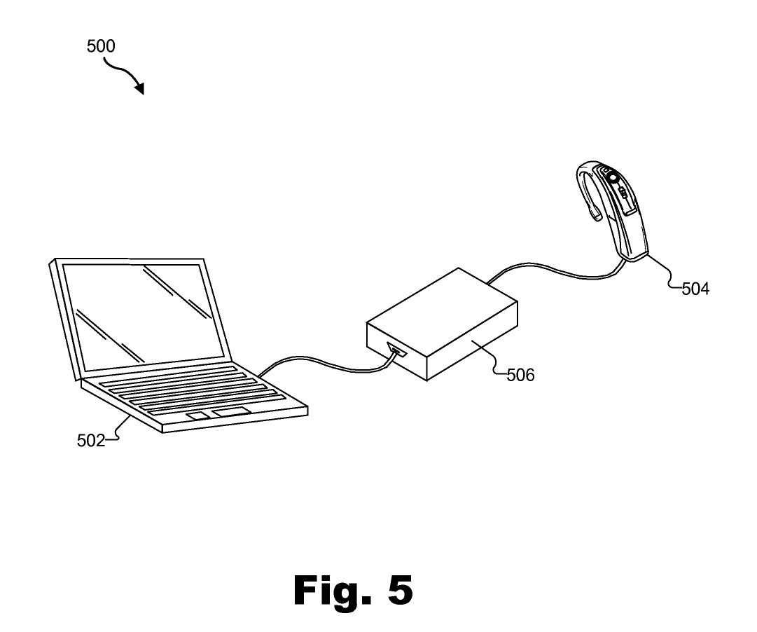

FIG. 5 illustrates an exemplary implementation 500 of fitting system 200. In implementation 500, a fitting station 502 may be selectively and communicatively coupled to a BTE unit 504 by way of a CPI device 506. BTE unit 504 is merely exemplary of the many different types of sound processors that may be used in accordance with the systems and methods described herein. Fitting station 502 may be selectively and communicatively coupled to any other type of sound processor as may serve a particular implementation.

Fitting station 502 may include any suitable computing device and/or combination of computing devices and may be configured to perform one or more of the fitting operations described herein. For example, fitting station 502 may display one or more GUIs configured to facilitate loading of one or more program sets onto BTE unit 504, selection of one or more programs by which BTE unit 504 operates, adjustment of one or more control parameters by which BTE unit 504 operates, and/or any other fitting operation as may serve a particular implementation. Fitting station 502 may be utilized by an audiologist, a clinician, and/or any other user to fit BTE unit 504 to a patient.

CPI device 506 may be configured to facilitate communication between fitting station 502 and BTE unit 504. In some examples, CPI device 506 may be selectively and communicatively coupled to fitting station 502 and/or BTE unit 504 by way of one or more ports included within fitting station 502 and BTE unit 504.

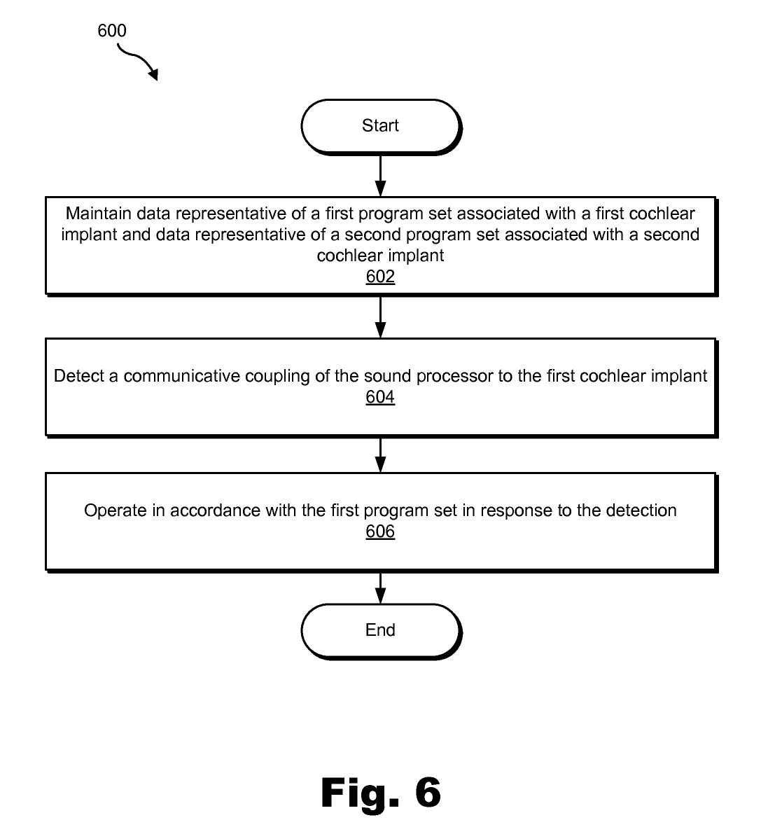

FIG. 6 illustrates an exemplary method 600 of operation of a bilateral sound processor. While FIG. 6 illustrates exemplary steps according to one embodiment, other embodiments may omit, add to, reorder, and/or modify any of the steps shown in FIG. 6. One or more of the steps shown in FIG. 6 may be performed by any component or combination of components of sound processor 104.

In step 602, a sound processor maintains data representative of a first program set associated with a first cochlear implant and data representative of a second program set associated with a second cochlear implant. For example, as described above, sound processor 104 may be configured to maintain first program set data 410 representative of a first program set associated with a first cochlear implant and second program set data 412 representative of a second program set associated with a second cochlear implant. In some examples, both the first and second program sets may be associated with a particular bilateral cochlear implant patient (e.g., the first program set may be associated with a first cochlear implant implanted in the patient and associated with a first ear of the patient and the second program set may be associated with a second cochlear implant implanted in the patient and associated with a second ear of the patient).

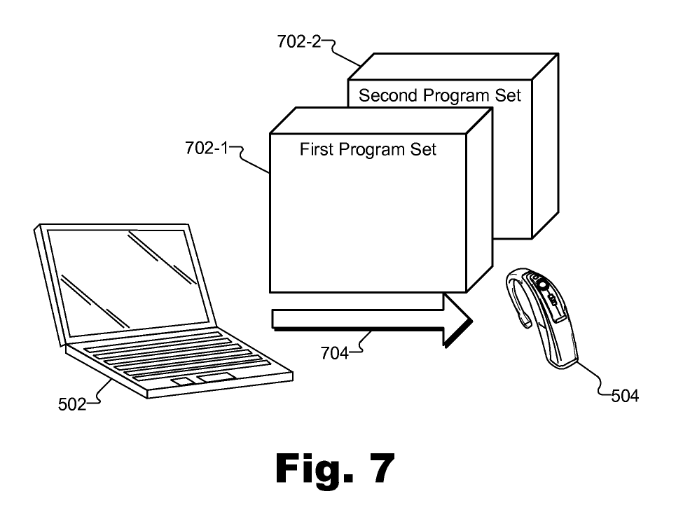

The first and second program sets may be loaded onto sound processor 104 and/or fitted to a corresponding patient using fitting subsystem 202. For example, FIG. 7 illustrates an exemplary loading of multiple program sets onto a sound processor during or prior to a fitting session in which the program sets are used to fit the sound processor to a patient. As shown in FIG. 7, first program set 702-1 and second program set 702-2 (collectively referred to as “program sets 702”) may be loaded onto BTE unit 504 by fitting station 502. The loading is represented in FIG. 7 by arrow 704. In some examples, the loading of program sets 702 onto BTE unit 504 may be performed by transmitting data representative of program sets 702 to BTE unit 504 and directing BTE unit 504 to cache the data as a library of program sets in a storage medium included within the sound processor.

Once programs sets 702 are loaded onto BTE unit 504, fitting station 502 may be utilized to fit BTE unit 504 to a patient. For example, an audiologist may use fitting station 502 and first program set 702-1 to fit BTE unit 504 to a first cochlear implant implanted in the patient and associated with a first ear of the patient. Similarly, the audiologist may use fitting station 502 and second program set 702-2 to fit BTE unit 504 to a second cochlear implant implanted in the patient and associated with a second ear of the patient. Accordingly, BTE unit 504 may store program sets 702 associated with and/or be fitted to both cochlear implants of a bilateral cochlear implant patient.

Returning to FIG. 6, in step 604, a communicative coupling of the sound processor to the first cochlear implant is detected. For example, detection facility 404 may be configured to detect that sound processor 104 is communicatively coupled to a first cochlear implant implanted in a bilateral cochlear implant patient.

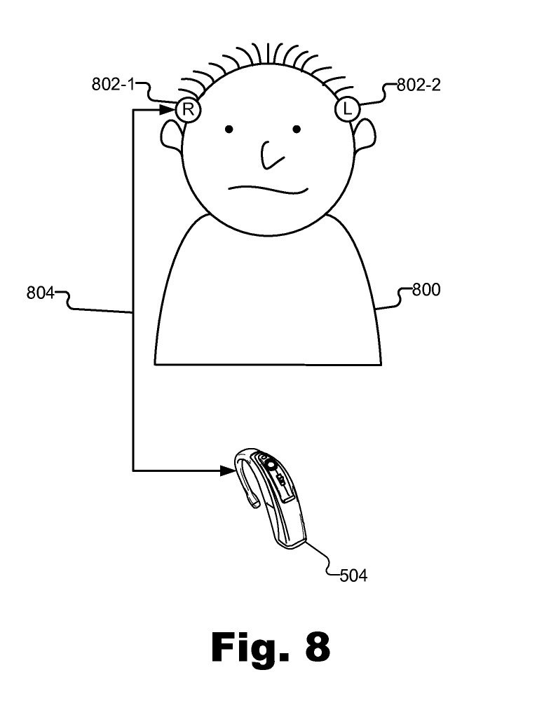

FIG. 8 illustrates an exemplary communicative coupling of a sound processor to a first cochlear implant implanted in a bilateral cochlear implant patient and associated with a first ear of the patient. As shown, a bilateral cochlear implant patient 800 (or simply “patient 800”) having a first cochlear implant 802-1 and a second cochlear implant 802-2 (collectively referred to as “cochlear implants 802”) may facilitate the communicative coupling of BTE unit 504 to first cochlear implant 802-1 by way of a communication link 804 (e.g., by placing BTE unit 504 behind patient's 800 right ear and positioning a corresponding headpiece to communicate with first cochlear implant 802-1).

BTE unit 504 may be configured to detect the communicative coupling with first cochlear implant 802-1 in any suitable manner. For example, BTE unit 504 may be configured to detect a transmission/receipt of signals to/from first cochlear implant 802-1.

In some examples, BTE unit 504 may be configured to identify the particular cochlear implant to which it is communicatively coupled. For example, BTE unit 504 may be configured to detect a first unique identifier (e.g., a first unique serial number) associated with first cochlear implant 802-1. In some examples, BTE unit 504 may be configured to receive data representative of the first unique identifier from first cochlear implant 802-1 and identify first cochlear implant 802-1 based on the first unique identifier. Upon receiving the first unique identifier, BTE unit 504 may compare the first unique identifier to unique identifier data maintained by BTE unit 504 to identify first cochlear implant 802-1, patient 800, and/or one or more programs sets associated with first cochlear implant 802-1 and/or patient 800.

Returning to FIG. 6, in step 606, the sound processor may operate in accordance with the first program set in response to the detecting of the communicative coupling of the sound processor to the first cochlear implant. As described above, for example, operation facility 406 of sound processor 104 may be configured to operate (e.g., process audio signals) in accordance with the first program set in response to a detection that sound processor 104 is communicatively coupled to the first cochlear implant.

Returning to FIG. 8, BTE unit 504 may be configured to operate in accordance with a first program set (e.g., first program set 702-1) associated with first cochlear implant 802-1 in response to a detection of the communicative coupling of BTE unit 504 to first cochlear implant 802-1. For example, BTE unit 504 may be configured to perform one or more signal processing heuristics on an audio signal presented to patient 800 in accordance with the sound processing program(s) and/or control parameters of the first program set. Accordingly, BTE unit 504 may be configured to dynamically adapt its operation based on the particular cochlear implant to which it is coupled. By so doing, BTE unit 504 may be configured to successfully operate in conjunction with a plurality of cochlear implants without the risk of overstimulation of one cochlear implant based on the control parameters and/or sound processing programs associated with another cochlear implant.

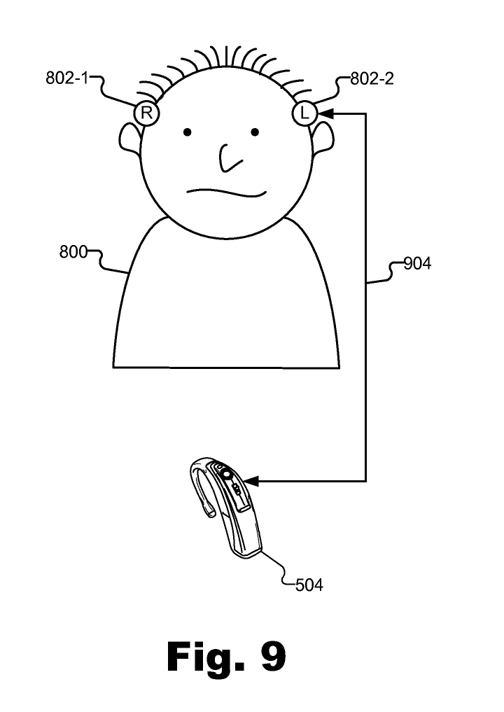

BTE unit 504 may be further configured to detect a communicative decoupling of BTE unit 504 from first cochlear implant 802-1 and a subsequent communicative coupling of BTE unit 504 to second cochlear implant 802-2. For example, as shown in FIG. 9, patient 800 can switch BTE unit 504 from first cochlear implant 802-1 to second cochlear implant 802-2 (e.g., by switching BTE unit 504 from the right ear to the left ear and positioning the corresponding headpiece to communicate with second cochlear implant 802-2). As a result, BTE unit 504 may communicatively decouple from first cochlear implant 802-1 and communicatively couple to second cochlear implant 802-2 by way of communication link 904.

BTE unit 504 may be configured to detect that communication with first cochlear implant 802-1 has been broken and that communication with second cochlear implant 802-2 has been established in any suitable manner. In some examples, BTE unit 504 may be configured to receive data representative of a second unique identifier associated with second cochlear implant 802-2 from second cochlear implant 802-2 and identify second cochlear implant 802-2 based on the second unique identifier.

In response to a detection of the communicative coupling of BTE unit 504 to second cochlear implant 802-2, BTE unit 504 may be configured to operate in accordance with a second program set (e.g., second program set 702-2) associated with second cochlear implant 802-2. For example, BTE unit 504 may be configured to perform one or more signal processing heuristics on an audio signal presented to patient 800 in accordance with the sound processing program(s) and/or control parameters of the second program set. Accordingly, BTE unit 504 may be configured to dynamically adapt to and operate in accordance with a communicative coupling to either of first cochlear implant 802-1 and second cochlear implant 802-2.

In certain embodiments, one or more of the components and/or processes described herein may be implemented and/or performed by one or more appropriately configured computing devices. To this end, one or more of the systems and/or components described above may include or be implemented by any computer hardware and/or computer-implemented instructions (e.g., software) embodied on a non-transitory computer-readable medium configured to perform one or more of the processes described herein. In particular, system components may be implemented on one physical computing device or may be implemented on more than one physical computing device. Accordingly, system components may include any number of computing devices, and may employ any of a number of computer operating systems.

In certain embodiments, one or more of the processes described herein may be implemented at least in part as instructions executable by one or more computing devices. In general, a processor (e.g., a microprocessor) receives instructions, from a tangible computer-readable medium, (e.g., a memory, etc.), and executes those instructions, thereby performing one or more processes, including one or more of the processes described herein. Such instructions may be stored and/or transmitted using any of a variety of known non-transitory computer-readable media.

A non-transitory computer-readable medium (also referred to as a processor-readable medium) includes any non-transitory medium that participates in providing data (e.g., instructions) that may be read by a computer (e.g., by a processor of a computer). Such a non-transitory medium may take many forms, including, but not limited to, non-volatile media and/or volatile media. Non-volatile media may include, for example, optical or magnetic disks and other persistent memory. Volatile media may include, for example, dynamic random access memory (“DRAM”), which typically constitutes a main memory. Common forms of non-transitory computer-readable media include, for example, a floppy disk, flexible disk, hard disk, magnetic tape, any other magnetic medium, a CD-ROM, DVD, any other optical medium, a RAM, a PROM, an EPROM, a FLASH-EEPROM, any other memory chip or cartridge, or any other non-transitory medium from which a computer can read.

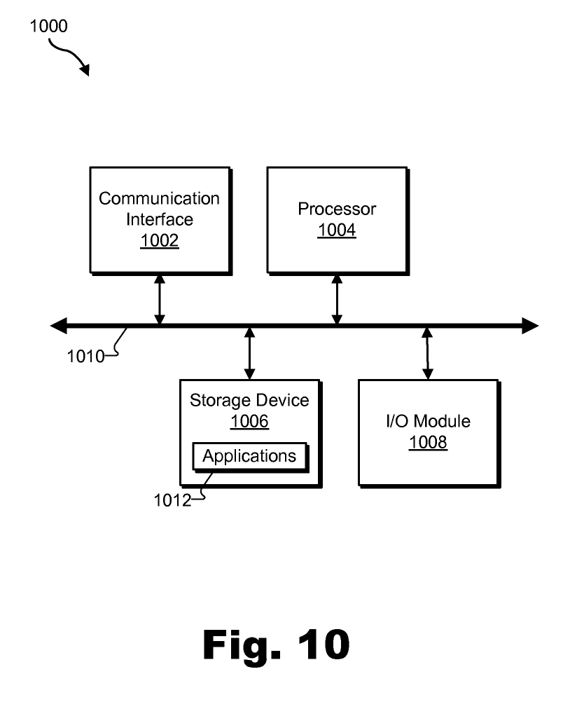

FIG. 10 illustrates an exemplary computing device 1000 that may be configured to perform one or more of the processes described herein. As shown in FIG. 10, computing device 1000 may include a communication interface 1002, a processor 1004, a storage device 1006, and an input/output (“I/O”) module 1008 communicatively connected via a communication infrastructure 1010. While an exemplary computing device 1000 is shown in FIG. 10, the components illustrated in FIG. 10 are not intended to be limiting. Additional or alternative components may be used in other embodiments. Components of computing device 1000 shown in FIG. 10 will now be described in additional detail.

Communication interface 1002 may be configured to communicate with one or more computing devices. Examples of communication interface 1002 include, without limitation, a wired network interface (such as a network interface card), a wireless network interface (such as a wireless network interface card), a modem, and any other suitable interface. Communication interface 1002 may additionally or alternatively provide such a connection through, for example, a local area network (such as an Ethernet network), a personal area network, a telephone or cable network, a satellite data connection, a dedicated URL, or any other suitable connection. Communication interface 1002 may be configured to interface with any suitable communication media, protocols, and formats, including any of those mentioned above.

Processor 1004 generally represents any type or form of processing unit capable of processing data or interpreting, executing, and/or directing execution of one or more of the instructions, processes, and/or operations described herein. Processor 1004 may direct execution of operations in accordance with one or more applications 1012 or other computer-executable instructions such as may be stored in storage device 1006 or another non-transitory computer-readable medium.

Storage device 1006 may include one or more data storage media, devices, or configurations and may employ any type, form, and combination of data storage media and/or device. For example, storage device 1006 may include, but is not limited to, a hard drive, network drive, flash drive, magnetic disc, optical disc, random access memory (“RAM”), dynamic RAM (“DRAM”), other non-volatile and/or volatile data storage units, or a combination or sub-combination thereof. Electronic data, including data described herein, may be temporarily and/or permanently stored in storage device 1006. For example, data representative of one or more executable applications 1012 (which may include, but are not limited to, one or more of the software applications described herein) configured to direct processor 1004 to perform any of the operations described herein may be stored within storage device 1006. In some examples, data may be arranged in one or more databases residing within storage device 1006.

I/O module 1008 may be configured to receive user input and provide user output and may include any hardware, firmware, software, or combination thereof supportive of input and output capabilities. For example, I/O module 1008 may include hardware and/or software for capturing user input, including, but not limited to, a keyboard or keypad, a touch screen component (e.g., touch screen display), a receiver (e.g., an RF or infrared receiver), and/or one or more input buttons.

I/O module 1008 may include one or more devices for presenting output to a user, including, but not limited to, a graphics engine, a display (e.g., a display screen, one or more output drivers (e.g., display drivers), one or more audio speakers, and one or more audio drivers. In certain embodiments, I/O module 1008 is configured to provide graphical data to a display for presentation to a user. The graphical data may be representative of one or more graphical user interfaces and/or any other graphical content as may serve a particular implementation.

In some examples, any of the facilities described herein may be implemented by or within one or more components of computing device 1000. For example, one or more applications 1012 residing within storage device 1006 may be configured to direct processor 1004 to perform one or more processes or functions associated with communication facility 302, user interface facility 304, fitting facility 306, program loading facility 308, communication facility 402, detection facility 404, and/or operation facility 406. Likewise, storage facility 310 and/or storage facility 408 may be implemented by or within storage device 1006.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A sound processor comprising:
a storage facility configured to maintain data representative of a first program set associated with a first cochlear implant and data representative of a second program set associated with a second cochlear implant;
a detection facility communicatively coupled to the storage facility and configured to detect when the sound processor is communicatively coupled to the first cochlear implant and to detect when the sound processor is communicatively coupled to the second cochlear implant; and
an operation facility communicatively coupled to the detection facility and configured to operate in accordance with the first program set in response to a detection by the detection facility that the sound processor is communicatively coupled to the first cochlear implant and to operate in accordance with the second program set in response to a detection by the detection facility that the sound processor is communicatively coupled to the second cochlear implant.

2. The sound processor of claim 1, wherein the detection facility is further configured to:
detect when the sound processor is communicatively coupled to the first cochlear implant by detecting a first unique identifier associated with the first cochlear implant; and
detect when the sound processor is communicatively coupled to the second cochlear implant by detecting a second unique identifier associated with the second cochlear implant.

3. The sound processor of claim 2, wherein the first unique identifier comprises a first unique serial number and the second unique identifier comprises a second unique serial number.

4. The sound processor of claim 3, wherein the first program set is further associated with the first unique serial number and the second program set is further associated with the second unique serial number.

5. The sound processor of claim 2, wherein the storage facility is further configured to maintain unique identifier data representative of the first and second unique identifiers and wherein the detection facility is further configured to compare the detected first and second unique identifiers to the unique identifier data.

6. The sound processor of claim 1, wherein the operations facility is further configured to process one or more audio signals in accordance with the first program set in response to a detection by the detection facility that the sound processor is communicatively coupled to the first cochlear implant and to process one or more audio signals in accordance with the second program set in response to a detection by the detection facility that the sound processor is communicatively coupled to the second cochlear implant.

7. The sound processor claim 1, wherein the sound processor comprises a behind-the-ear (“BTE”) unit.

8. A sound processor comprising:
a storage facility configured to maintain data representative of a first program set associated with a first cochlear implant and data representative of a second program set associated with a second cochlear implant;
a communication facility communicatively coupled to the storage facility and configured to selectively communicate with the first cochlear implant and the second cochlear implant;
a detection facility communicatively coupled to the communication facility and configured to detect when the sound processor is communicatively coupled to the first cochlear implant and to detect when the sound processor is communicatively coupled to the second cochlear implant; and
an operation facility communicatively coupled to the detection facility and configured to process one or more audio signals in accordance with the first program set in response to a detection by the detection facility that the sound processor is communicatively coupled to the first cochlear implant and to process one or more audio signals in accordance with the second program set in response to a detection by the detection facility that the sound processor is communicatively coupled to the second cochlear implant.

9. The sound processor of claim 8, wherein the detection facility is further configured to:
detect a first unique identifier associated with the first cochlear implant and a second unique identifier associated with the second cochlear implant; and
identify the first cochlear implant based on the first unique identifier and the second cochlear implant based on the second unique identifier.

10. The sound processor of claim 9, wherein the first unique identifier comprises a first unique serial number and the second unique identifier comprises a second unique serial number.

11. The sound processor of claim 10, wherein the first program set is further associated with the first unique serial number and the second program set is further associated with the second unique serial number.

12. A method comprising:
maintaining, by a sound processor, data representative of a first program set associated with a first cochlear implant and data representative of a second program set associated with a second cochlear implant;
detecting, by the sound processor, a communicative coupling of the sound processor to the first cochlear implant; and
operating, by the sound processor in response to the detecting, in accordance with the first program set.

13. The method of claim 12, wherein the detecting the communicative coupling of the sound processor to the first cochlear implant comprises at least one of detecting a successful transmission of a signal from the sound processor to the first cochlear implant and detecting a receipt of a signal by the sound processor from the first cochlear implant.

14. The method of claim 12, wherein the operating comprises processing at least one audio signal in accordance with the first program set.

15. The method of claim 12, wherein the detecting of the communicative coupling of the sound processor to the first cochlear implant comprises:
detecting a first unique identifier associated with the first cochlear implant; and
identifying the first cochlear implant based on the first unique identifier.

16. The method of claim 15, wherein the first unique identifier comprises a first unique serial number.

17. The method of claim 12, further comprising:
detecting, by the sound processor, a communicative decoupling of the sound processor from the first cochlear implant;
detecting, by the sound processor, a communicative coupling of the sound processor to the second cochlear implant; and
operating, by the sound processor in response to the detecting of the communicative coupling of the sound processor to the second cochlear implant, in accordance with the second program set.

18. The method of claim 17, wherein the operating in accordance with the second program set comprises processing at least one audio signal in accordance with the second program set.

19. The method of claim 17, wherein the detecting of the communicative coupling of the sound processor to the second cochlear implant comprises:
detecting a second unique identifier associated with the second cochlear implant; and
identifying the second cochlear implant based on the second unique identifier.

20. The method of claim 12, further comprising receiving, by the sound processor, the data representative of the first program set and the data representative of the second program set from a fitting station.
